(19) United States
(12) Patent Application Publication
(10) Pub. No.: US 2013/0288917 A1
(43) Pub. Date: Oct. 31, 2013

(54) Rapid High Resolution, High Throughput RNA Structure, RNA-Macromolecular Interaction, and RNA-Small Molecule Interaction Mapping

(71) Applicants: Jeffrey S. Glenn, Palo Alto, CA (US); Phillip S. Pang, Palo Alto, CA (US); Shripa Patel, Stanford, CA (US)

(72) Inventors: Jeffrey S. Glenn, Palo Alto, CA (US); Phillip S. Pang, Palo Alto, CA (US); Shripa Patel, Stanford, CA (US)

(73) Assignee: The Board of Trustees of the Leland Stanford Junior University, Palo Alto, CA (US)

(21) Appl. No.: 13/880,314

(22) PCT Filed: Oct. 21, 2011
(86) PCT No.: PCT/US11/57232

§ 371 Date: Jul. 2, 2013

Related U.S. Application Data
(60) Provisional application No. 61/455,662, filed on Oct. 21, 2010.

(51) Int. Cl. C12Q 1/70

(52) U.S. Cl. CPC C12Q 1/707; G06F 19/22
USPC 506/9; 435/6.12; 435/5; 435/6.11; 702/20

(57) ABSTRACT

Compositions and methods are provided for a rapid, high-resolution, high-throughput method for determining the intramolecular interactions between the nucleotides present in an polynucleotide, such that single-stranded nucleotides, and nucleotides in a double-stranded configuration are distinguished and identified. Tertiary contacts and solvent accessible regions may also be determined, where such contacts and regions may result from a single stranded configuration, intermolecular interactions with other macromolecules including, without limitation, DNA, protein, RNA, etc.; intermolecular interactions with small molecules which may include drug candidates; or a combination of macromolecules and small molecules.

### BACKGROUND OF THE INVENTION

RNA is a ubiquitous macromolecule that plays numerous, critical roles in biology. Some examples include its role as a structural scaffold (e.g. ribosome), as a messenger (e.g. messenger RNA), as a catalytic agent (e.g. ribozymes), in cell regulation (e.g. microRNA), in translation (i.e. IRES), as well as serving as the genome for numerous pathogens (e.g. RNA viruses).

Recent discoveries have shown that RNA plays a larger role in biology than previously realized, for example in posttranscriptional regulation, development, immunity, and peptide bond formation. The functional form of single stranded RNA molecules, just like proteins, frequently requires a specific tertiary structure. The scaffold for this structure is provided by hydrogen bonding within the molecule. This leads to several recognizable “domains” of secondary structure like hairpin loops, bulges and internal loops.

It is necessary to determine the native structures of RNAs to understand their mechanisms of action, and determining secondary structure is a crucial step in this process. RNA secondary structure can be predicted by free energy minimization with nearest neighbor parameters to evaluate stability. Nuclease cleavage data can be used to refine structure prediction and improve accuracy. A predicted secondary structure can guide further experiments or comparative sequence analysis and also aid in the design of RNA molecules.

Chemical modification is a technique that reveals solvent accessible nucleotides. The nucleotides accessible to 1-cyclohexyl-3-(2-morpholinoethyl) carbodiimide metho-p-toluene sulfonate, dimethyl sulfate, and kethoxal are unpaired, in A-U or G-C pairs at helix ends, in G-U pairs anywhere, or adjacent to G-U pairs. This limited specificity differs from that observed with nucleases. Methods for analysis of RNA secondary structure include those described by Mortimer & Weeks (2009) Nature protocols 4, 1413-1421; and Wilkinson et al. (2006) Nature protocols 1, 1610-1616.

With in vitro mapping and ex virio mapping there exists the possibility of introducing artifacts during the folding or extracting process. By contrast, in vivo mapping would allow for biologically relevant RNA secondary structure to be deduced with greater certainty, from even a small number of cells. It also allows for RNA secondary structure as it exists in specific cell compartments, viruses, etc. to be mapped. The present invention addresses this issue.

### SUMMARY OF THE INVENTION

Compositions and methods are provided for a rapid, high-resolution, high-throughput method for determining the intramolecular interactions between the nucleotides present in an RNA polymer, such that single-stranded nucleotides, and nucleotides in a double-stranded configuration are distinguished and identified. Tertiary contacts and solvent accessible regions may also be determined, where such contacts and regions may result from a single stranded configuration, intermolecular interactions with other macromolecules including, without limitation, DNA, protein, RNA, etc.; intermolecular interactions with small molecules which may include drug candidates; or a combination of macromolecules and small molecules. The methods of the invention are not limited in ability to predict RNA secondary structure, but are also sensitive to RNA tertiary structure, and can distinguish between non-base paired, yet stacked nucleotides and non-base paired nucleotides that are flipped out of helices, thus providing information on the character of internal RNA bulges and loops.

In the methods of the invention a population of RNA molecules are contacted with a modifying agent. The modified RNAs are utilized as a template for polymerization, e.g. with a reverse transcriptase enzyme. The population of polymerization products are ligated at the 3′ end to an adapter, which provides a site for amplification. The amplification products are then sized, where each termination point represents a site that the modifying agent acted on the initial RNA, indicating the presence of a secondary structure feature, e.g. a solvent accessible site, etc.

In some embodiments the RNA population is present in an intact cell or a viral particle. In some embodiments the RNA population is contacted with an agent, e.g. a drug candidate, interacting protein, membrane, etc. prior to contacting with the modifying agent; where the resulting secondary structure is compared to the secondary structure information obtained in the absence of the agent. In some such embodiments a library of drug candidates are screening for the ability to alter secondary structure. In some such embodiments an RNA of interest is initially allowed to interact with a macromolecule of interest prior to contacting with a drug candidate, e.g. macromolecules such as viral coat proteins; replicative, transcriptional or translational proteins; and the like. Drug candidates identified as altering secondary structure may be further screened for the ability to alter biological functions of the RNA, e.g. alteration of translation, alteration of viral replication, alteration of viral packaging, and the like.

In some embodiments the RNA population is subjected to direct or random mutagenesis prior to contacting with the modifying agent, where the secondary structure of the mutant RNA may be compared to the secondary structure of a corresponding control RNA.

In some embodiments of the invention, the RNA of interest is a viral RNA. In some such embodiments the viral RNA is a viral genome. In other such embodiments the viral RNA is a viral mRNA. In other embodiments, a single stranded DNA virus is probed for secondary structure, where the single stranded DNA is substituted for RNA in the methods of the invention. In some embodiments the viral genome is an influenza virus genome. In some embodiments the viral genome is a Hepatitis C genome.

These and other advantages, and features of the invention will become apparent to those persons skilled in the art upon reading the details of the compositions and methods of use are more fully described below.

### BRIEF DESCRIPTION OF THE DRAWINGS

FIG. 1. sf-SHAPE-determined Secondary Structure Model of the HCV IRES. a. First proposed Domain I-Domain II structure, as determined by enzymatic mapping₁₅. Nucleotides in the miR-122 target region cleaved by single-stranded nucleases (red). b. Regions of the HCV IRES structure resolved by NMR and crystallography (blue). c. An HCV IRES composed of NTs 1-377 is sensitive to miR-122 knockdown. Luciferase translation assay in huh 7.5 hepatoma cells. Error bars denote the standard deviation calculated from 9 replicates. d. sf-SHAPE reactivities mapped into the IRES structure. Prior proposed structure for the apical loop of D-III is shown₁₅ (upper right corner).

FIG. 2. The first 30 nucleotides of the 5′UTR of HCV form a potential triple-stranded RNA motif (triplex). a. Sequence logo depicting the pyrimidine-purine-pyrimidine motif in the first 30 NTs of HCV, based on an alignment from >300 sequences, representing all 6 genotypes. b. Hypothesized triplex structure causing reverse transcriptase pausing. c. Electrophoretic trace of various IRES mutations. Mutation of the stem of domain I (light blue) or the first miR-122 target site (red), but not the loop (pink), markedly reduces RT pausing in the region of the first miR-122 target site. d. Disruption of the triplex by mutation of the domain I stem sequence reduces translation, but remains sensitive to miR-122 knockdown. Luciferase translation assay in huh 7.5 hepatoma cells. Error bars denote the standard deviation calculated from 9 replicates.

FIG. 3. The tail of miR-122 makes non-canonical interactions with its target site and alters the conformation of the AUG start site (domain IV). a. TOP: sf-SHAPE reactivities for the HCV IRES (WT, red), and the IRES in the presence of miR-122 (green) and control miR-124 (grey). BOTTOM: SHAPE reactivity for the HCV IRES in the presence of miR-122 minus the SHAPE reactivity for the IRES in the absence of miR-122 (purple); miR-124 difference trace (orange). b. Electrophoretic traces for the HCV IRES folded and acylated by NMIA (which detects flexible nucleotides), in the absence (red) and presence (green) of miR-122. The control trace (black): unmodified HCV IRES. miR-122 seed site (grey open letters); Non-seed, canonical base-pairs (black); Nonseed, non-canonical base pairs (orange). c. LEFT: SHAPE reactivities for the second miR-122 target site. The mutant miR-122 with the tail of miR-124 binds to the complementary seed site on HCV, but does not make non-canonical target interactions. RIGHT: The tail region of miR-122 is required to enhance the flexibility of nucleotides near the AUG start site (domain IV).

FIG. 4. A Model for the conformational change induced by miR-122 at the AUG start site (domain IV). miR-122 binds at the target sites between domains I and II, through both canonical and non-canonical interactions. This binding destabilizes the domain IV stem, thereby enhancing AUG start site accessibility by the ribosome.

FIG. 5A-F. High-throughput, high resolution RNA secondary structure determination.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a peptide” includes a plurality of such peptides and reference to “the inhibitor” includes reference to one or more inhibitors and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

RNA Modifying Agent. RNA modifying agents include any chemical or enzymatic agent that alters the RNA at a specific location, where the location is determined by the secondary structure of the RNA. The modification is such that it causes a halt in a polymerization reaction when the modified RNA is used as a template for the polymerization. For example, agents are known in the art that modify the 2′OH of nucleotides that have structural flexibility, with higher flexibility correlating with single stranded regions in the structure.

Agents of interest include, without limitation, chemical agents such as N-methylisatoic anhydride (NMIA), 1-methyl-7-nitroisatoic anhydride (1M7), Benzoyl CN, dimethylsulfoxide (DMSO), diethylpyrocarbonate (DEPC), Pb²⁺, Fe²⁺, etc.; and enzymatic agents, e.g. RNAse T1, cobra venome V1 nuclease, etc. These chemical or enzymatic modifications probe the structure of the RNA by cleaving or chemically altering the RNA in known ways. Chemical agents find use in structure determination within the context of a cell or virion, whereas enzymatic means will generally require RNA extraction and refolding of the RNA.

RNA. As used herein the term RNA refers to oligo- or polynucleotides, usually at least partially single stranded, that are fully or partially comprised of ribonucleotides. RNA molecules of interest include, without limitation, microRNAs, siRNA, RNAi, shRNA, mRNA, tRNA, rRNA, viral RNA genomes, etc. RNAs of interest are usually of a sufficient length to have a secondary structure, e.g. at least about 10 nt in length.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

Virus RNA. Viral RNAs are of interest for secondary structure mapping. Viral RNAs of interest include mRNA from any viral genome, and genomic RNA from those viruses whose genomes have significant secondary structure, particularly single stranded RNA and DNA viruses, although certain double-stranded virus genomes may have specific regions with significant secondary structure.

Viruses of interest include, without limitation, positive-sense ssRNA viruses, e.g. Arteriviridae; Coronaviridae, includes coronavirus, SARS; Roniviridae; Dicistroviridae; Iflaviridae; Marnaviridae; Picornaviridae, including poliovirus, cold virus, Hepatitis A virus; Secoviridae; Alphaflexiviridae; Betaflexiviridae; Gammaflexiviridae; Tymoviridae; Astroviridae; Barnaviridae; Bromoviridae; Caliciviridae, including Norwalk virus; Closteroviridae; Flaviviridae, including Yellow fever virus, West Nile virus, Hepatitis C virus, Dengue fever virus; Leviviridae; Luteoviridae; Narnaviridae; Nodaviridae; Potyviridae; Tetraviridae; Togaviridae, including Rubella virus, Ross River virus, Sindbis virus, Chikungunya virus; Benyvirus; Furovirus; Hepevirus, including Hepatitis E virus; Hordeivirus; ldaeovirus; Ourmiavirus; Pecluvirus; Pomovirus; Sobemovirus; Tobamovirus, including tobacco mosaic virus; Tobravirus; Umbravirus.

Negative-sense ssRNA viruses include, without limitation, Bornaviridae, including Borna disease virus; Filoviridae, including Ebola virus, Marburg virus; Paramyxoviridae, including Measles virus, Mumps virus, Nipah virus, Hendra virus; Rhabdoviridae, including Rabies virus; Arenaviridae, including Lassa virus; Bunyaviridae, including Hantavirus, Crimean-Congo hemorrhagic fever; Orthomyxoviridae, including Influenza viruses; Deltavirus; Nyavirus; Ophiovirus; Tenuivirus; Varicosavirus.

Detectable Label. For monitoring the length of an amplification product, a convenient method is to label a molecule with a detectable moiety, which may be fluorescent, luminescent, radioactive, etc. Fluorescent moieties are readily available for labeling virtually any biomolecule, structure, or cell type. Fluorescence technologies have matured to the point where an abundance of useful dyes are now commercially available. These are available from many sources, including Sigma Chemical Company (St. Louis Mo.) and Molecular Probes (Handbook of Fluorescent Probes and Research Chemicals, Seventh Edition, Molecular Probes, Eugene Oreg.). Other fluorescent sensors have been designed to report on biological activities or environmental changes, e.g. pH, calcium concentration, electrical potential, proximity to other probes, etc. Methods of interest include calcium flux, nucleotide incorporation, quantitative PAGE (proteomics), etc.

Highly luminescent semiconductor quantum dots (zinc sulfide-capped cadmium selenide) have been covalently coupled to biomolecules for use in ultrasensitive biological detection (Stupp et al. (1997) Science 277(5330):1242-8; Chan et al. (1998) Science 281(5385):2016-8). Compared with conventional fluorophores, quantum dot nanocrystals have a narrow, tunable, symmetric emission spectrum and are photochemically stable (Bonadeo et al. (1998) Science 282(5393):1473-6). The advantage of quantum dots is the potential for exponentially large numbers of independent readouts from a single source or sample.

Amplification as used herein refers to an iterative process by which a nucleic acid is copied. Suitable methods for amplification include without limitation polymerase chain reaction, ligase chain reaction, strand displacement amplification, nucleic acid single base amplification, and transcription mediated amplification.

Amplification primer. The term “primer,” as used herein refers to an isolated oligonucleotide which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer, use of the method, and the parameters used for primer design, as disclosed herein.

Adapter: a short oligonucleotide of sufficient length to allow priming of an amplification reaction, e.g. at least about 8 bases in length, at least about 10, at least about 12, or more, which is ligated to a single stranded polynucleotide.

As used herein the term “isolated,” when used in the context of an isolated compound, refers to a compound of interest that is in an environment different from that in which the compound naturally occurs. “Isolated” is meant to include compounds that are within samples that are substantially enriched for the compound of interest and/or in which the compound of interest is partially or substantially purified. For example, an isolated peptide of the invention is at least 60%, by weight, free from the proteins and naturally-occurring organic molecules with which it is naturally associated or, in the context of synthetic peptides, at least 60% by weight free of synthetic peptides of different sequence and intermediates. Preferably, the preparation is at least 75%, more preferably at least 90%, and most preferably at least 99%, by weight, peptide. An isolated peptide as described herein may be obtained, for example, by chemically synthesizing the protein or peptide, or by expression of a recombinant nucleic acid encoding a peptide of interest, with chemical synthesis likely being preferred. Purity can be measured by any appropriate method, e.g., column chromatography, mass spectrometry, HPLC analysis, and the like.

The terms “active agent,” “antagonist”, “inhibitor”, “drug” and “pharmacologically active agent” are used interchangeably herein to refer to a chemical material or compound which, when administered to an organism (human or animal) induces a desired pharmacologic and/or physiologic effect by local and/or systemic action.

As used herein, the terms “treatment,” “treating,” and the like, refer to obtaining a desired pharmacologic and/or physiologic effect, such as reduction of viral titer. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease (as in liver fibrosis that can result in the context of chronic HCV infection); (b) inhibiting the disease, i.e., arresting its development; and (c) relieving the disease, i.e., causing regression of the disease (e.g., reduction in viral titers).

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the terms “determining,” “measuring,” “assessing,” and “assaying” are used interchangeably and include both quantitative and qualitative determinations.

A “therapeutically effective amount” or “efficacious amount” means the amount of a compound that, when administered to a mammal or other subject for treating a disease, condition, or disorder, is sufficient to effect such treatment for the disease, condition, or disorder. The “therapeutically effective amount” will vary depending on the compound, the disease and its severity and the age, weight, etc., of the subject to be treated.

The term “unit dosage form,” as used herein, refers to physically discrete units suitable as unitary dosages for human and animal subjects, each unit containing a predetermined quantity of a compound (e.g., an aminopyrimidine compound, as described herein) calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

### Methods of the Invention

Methods are provided for high-throughput analysis of polynucleotide secondary structure, where the polynucleotide may be any polynucleotide that is at least partially single-stranded, usually an RNA. RNAs of interest may be obtained from any source, and can be probed in a cell-free extract, including isolated forms of the RNA, complex populations of RNAs; isolated or complex populations of RNAs in combination with other macromolecules, such as proteins, ribosomes, DNA, RNA, lipids, membranes, and the like; RNAs present in intact cells, intact virus particles, and the like.

The methods of the invention initially probe RNA structure by contacting an RNA of interest with a modifying agent, which modifying agent may include, without limitation chemical and enzymatic agents. The modifying agent cleaves or chemically alters the RNA in known ways, e.g. by modifying nucleotides that have structural flexibility. The modification results in truncation of a polymerase reaction, including without limitation reverse transcriptase, at the site of modification. In some embodiments the modifying agent is used to contact RNA present in an intact cell or virion, for which methods a chemical agent is preferred.

The modified RNA is then contacted with reverse transcriptase, under conditions permissive for transcription. In some embodiments the RT is primed with a primer specific for the RNA of interest. At each site of modification the transcription reaction is truncated, resulting in the generation of a set of DNA molecules whose length and sequence correspond to structural regions of interest. The single stranded DNA products of the reverse transcriptase are ligated to an adapter that provides a common sequence for amplification. The adapter provides an important function, as the sequences at the 3′ ends of the DNA molecules are often unknown. Optionally the adapter further comprises additional functionality, e.g. a site for initiation of transcription, a primer for sequencing, and the like.

The ligation products are amplified by any suitable means, utilizing a pair of primers. One of the primers specifically binds to the adapter sequence. The second primer is selected to bind to a region of interest in the RNA. Optionally the second primer binds to the site recognized by the reverse transcriptase primer, although other sites of interest may be selected. In some embodiments of the invention, a fluorophore is attached to an amplification primer.

Following amplification the size and/or sequence content of the amplification products is determined. Various methods known in the art find use for this purpose, including capillary electrophoresis, sequencing, gel electrophoresis, and the like. The lengths of product indicate each point in the molecule where there is a nucleotide accessible to modification. Based on the mapping of accessible nucleotides, a map can be built of the secondary structure.

Commercially available capillary electrophoresis machines are often used to sequence DNA. The sequencing procedure to do so requires tags (e.g. fluorophores) of different colors and mobility data for the specific tags. A preferred method of the invention utilizes an adaptation of what is commonly known as fragment analysis, which has been most commonly used for DNA fingerprinting.) Fragment analysis is optimized to determine the length and intensity of nucleic acid fragments. CE/Fragment analysis is used to determine the length and relative number of each fragment, which corresponds to the location and extent of modification at that location, for the target RNA under study.

The output of fragment analysis software is typically a list of fragments, their length, and their integrated peak area (which corresponds to their relative concentration). This data may often need to be processed, in order to correct for (a) fragments not due to modification (i.e. locations of non-specific RNA degradation, i.e. determined through the use of a control that consists of non-modified RNA) and (b) signal decay as a function of fragment length, which can be due to a number of factors including, but not limited to, processivity effects due to the RT, electrokinetic injection (EI) by the capillary electrophoresis machine (EI favors shorter over longer fragments), and the presence of possible multiple modifications to a given RNA molecule (e.g. multiple hits).

This signal decay can be corrected for by dividing the signal of each fragment length by a correction factor (X), that is the result of exponential function such as: X=A(p)^{length}+B (eq. 1). While the constant p in the above equation can be determined manually by inspection of its effect on the signal, this can lead to error and operator bias. It is also time consuming. An automated solution is to use a method we call an iterative inverse least-squares fit (IILSF).

The IILSF method is a method for detecting systematic bias in the signal. If for example, shorter fragments are over-represented compared to longer fragments, this would lead to a systematic bias such that the signals of shorter molecules are stronger than longer molecules. This bias can be represented by plotting peak areas (y-axis) as a function of fragment length (x-axis), and fitting this data to a simple line (y=m×+b). If the slope (m) deviates from zero, this suggests systematic bias. For example, if the slope is negative, this suggests that shorter molecules are over-represented compared to longer molecules. The common method for determining parameters m and b is known as a least-squares fit.

The goal of IISLF is to find a p value, which, when input into equation 1, results in slope m ˜0. P can thus be calculated empirically, by inputting an arbitrary p value into equation 1, and determining X=f(length). Peak areas are then divided by X_{length} and slope m is determined. This process is then iterated, choosing increasing (or decreasing) p values, until a slope near zero is found.

Every experimental data set is usually accompanied by a control data set, which consists of RNA that is unmodified. To determine the true reactivity of every position in the target RNA, the control values for each position must be subtracted from the experimental values for each position. However, small differences in the amount of initial RNA, modified RNA, or reverse transcribed DNA, may result from human error, processing artifacts, or during the CE process. These small differences result in systematic bias, such that the control or experimental data may be “offset” relative to one another, and thus require normalization. This normalization process can be carried out determined manually by inspection, but this can lead to error and operator bias. It is also time consuming. An automated method to perform this normalization, is provided, herein termed Minimization of the Median (MM).

In this method, the differences between all experimental positions and control positions are calculated, and the median of these differences determined. The control position is then iteratively multiplied by a range of correction factors (N), until the N which results in the smallest absolute median of the differences is determined.

### Screening Methods

In some embodiments of the invention, the RNA population is contacted with an agent, e.g. a drug candidate, interacting protein, membrane, etc. prior to contacting with the modifying agent; where the resulting secondary structure is compared to the secondary structure information obtained in the absence of the agent. In some such embodiments a library of drug candidates are screening for the ability to alter secondary structure. In some related embodiments an RNA of interest is initially allowed to interact with a macromolecule of interest prior to contacting with a drug candidate, e.g. macromolecules such as viral coat proteins; replicative, transcriptional or translational proteins; and the like.

In some embodiments the RNA population is subjected to direct or random mutagenesis prior to contacting with the modifying agent, where the secondary structure of the mutant RNA may be compared to the secondary structure of a corresponding control RNA. Such mutagenized RNAs can also be utilized in screening assays as described above.

Drug candidates identified as altering secondary structure may be further screened for the ability to alter biological functions of the RNA, e.g. alteration of translation, alteration of viral replication, alteration of viral packaging, and the like. Thus, in some embodiments, a test agent of interest inhibits an RNA function of interest by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90%, or more, compared to the level of function in the absence of the test agent.

A variety of different test agents may be screened using a subject method. Candidate agents encompass numerous chemical classes, e.g., small organic compounds having a molecular weight of more than 50 daltons and less than about 10,000 daltons, less than about 5,000 daltons, or less than about 2,500 daltons. Test agents can comprise functional groups necessary for structural interaction with proteins, e.g., hydrogen bonding, and can include at least an amine, carbonyl, hydroxyl or carboxyl group, or at least two of the functional chemical groups. The test agents can comprise cyclical carbon or heterocyclic structures and/or aromatic or polyaromatic structures substituted with one or more of the above functional groups. Test agents are also found among biomolecules including peptides, saccharides, fatty acids, steroids, purines, pyrimidines, derivatives, structural analogs or combinations thereof.

Test agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Moreover, screening may be directed to known pharmacologically active compounds and chemical analogs thereof, or to new agents with unknown properties such as those created through rational drug design.

In some embodiments, test agents are synthetic compounds. A number of techniques are available for the random and directed synthesis of a wide variety of organic compounds and biomolecules, including expression of randomized oligonucleotides. See for example WO 94/24314, hereby expressly incorporated by reference, which discusses methods for generating new compounds, including random chemistry methods as well as enzymatic methods.

In another embodiment, the test agents are provided as libraries of natural compounds in the form of bacterial, fungal, plant and animal extracts that are available or readily produced. Additionally, natural or synthetically produced libraries and compounds are readily modified through conventional chemical, physical and biochemical means. Known pharmacological agents may be subjected to directed or random chemical modifications, including enzymatic modifications, to produce structural analogs.

In some embodiments, the test agents are organic moieties. In this embodiment, as is generally described in WO 94/243 14, test agents are synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or candidate agents which can then be tested using the present invention.

As used herein, the term “determining” refers to both quantitative and qualitative determinations and as such, the term “determining” is used interchangeably herein with “assaying,” “measuring,” and the like.

In some embodiments, in addition to determining the effect of a test agent on RNA secondary structure, test agents are assessed for any cytotoxic activity it may exhibit toward a living eukaryotic cell, using well-known assays, such as trypan blue dye exclusion, an MTT (3-(4,5-dimethylthiazol-2-yl)-2,5-diphenyl-2H-tetrazolium bromide) assay, and the like. Agents that do not exhibit significant cytotoxic activity are considered candidate agents.

A variety of other reagents may be included in the screening assay. These include reagents like salts, neutral proteins, e.g. albumin, detergents, etc., including agents that are used create or modify secondary structure and/or reduce non-specific or background activity. Reagents that improve the efficiency of the assay, such as protease inhibitors, nuclease inhibitors, anti-microbial agents, etc. may be used. The components of the assay mixture are added in any order that provides for the requisite activity.

Assays of the invention include controls, where suitable controls include a sample in the absence of the test agent. Generally a plurality of assay mixtures is run in parallel with different agent concentrations to obtain a differential response to the various concentrations. Typically, one of these concentrations serves as a negative control, i.e. at zero concentration or below the level of detection.

The above-discussed compositions can be formulated using well-known reagents and methods. Compositions are provided in formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, the agent is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

The therapeutic agent(s) may administered in a unit dosage form and may be prepared by any methods well known in the art. Such methods include combining the compounds of the present invention with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g. PEG, are also good carriers.

Any drug delivery device or system that provides for the dosing regimen of the instant invention can be used. A wide variety of delivery devices and systems are known to those skilled in the art.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### Example 1

### Structural Insights into the Mechanism of microRNA Modulated Viral Translation

MicroRNAs (miRNAs) are small non-coding regulatory RNAs that control a vast array of cellular processes by repressing mRNA translation. Liver-expressed miR-122 is a miRNA that has been co-opted by hepatitis C virus (HCV) to enhance viral translations. Recently, miR-122 antagomir therapy in non-human primates has been shown to suppress HCV viremia. However, the mechanism by which miR-122 modulates HCV translation is unclear. To examine the structural changes that miR-122 exerts on the HCV internal ribosomal entry site (IRES), we developed an advanced SHAPE-based method of analysing RNA architecture. Using this method, we show that binding of miR-122 to one of its target sites within the 5′ UTR of HCV induces a conformational change in the HCV IRES at the distant AUG translation start site. Surprisingly, binding of miR-122 to this target site is mediated by a number of non-canonical base-pairings. Mutation of the 3′ half of miR-122 (tail) disrupted these non-canonical interactions and its ability to induce a conformational change at the AUG start site. We also observed that, in vitro, the other miR-122 target site in the 5′UTR of HCV is likely part of a triple stranded RNA motif. These results provide the first demonstration that the tail of this liver-encoded miRNA can directly alter the RNA conformation of the HCV IRES, and thereby provide new insights into the mechanism by which miR-122 enhances viral translation.

miRNAs are small endogenous RNAs that post-transcriptionally repress cellular protein expression by binding to the 3′ untranslated regions (UTRs) of mRNAs. Specificity is miated in large part by canonical base-pairings between the first 2-8 nucleotides in he miRNA (seed site), and complementary nucleotides (target sites) in 3′ UTRs. Hepatitis C virus (HCV), which replicates in liver cells, has co-opted the most abundant liver-expressed miRNA, miR-122, for its own purposes. Two genetically-validated miR-122 target sites have been identified within the HCV 5′ UTR. Binding of miR-122 to these sites enhances viral translation and replication. The potential clinical utility of this observation has been demonstrated by the use of miR-122 specific antagomirs, short nucleic acids complementary to miR-122, which reduce the effective concentration of miR-122. Such miRNA knockdown has been shown to decrease HCV viral titers in non-human primates.

The mechanism by which miR-122 enhances viral translation, however, has not been well-defined. Binding of miR-122 to its target sites in HCV has been shown to enhance formation of a complex consisting of the HCV IRES and the 48S ribosomal subunits. Thus, one possibility is that miR-122 targets translation-enhancing protein factors to the HCV IRES, similar to the role miRNAs play in targeting the RNA induced silencing complex (RISC) to the UTRs of cellular mRNAs. Another nonmutually exclusive possibility is that miR-122 directly alters the conformation of the HCV IRES, thereby enhancing the ability of the IRES to properly engage the ribosome.

Here we have resolved the secondary structure of the 5′UTR of HCV in the presence and absence of miR-122 in order to demonstrate that miR-122 induces a conformational change in the HCV IRES. Our analysis also reveals the importance of non-canonical base pairings for miRNA effector function, a previously unappreciated role for the tail region of miRNAs, and novel structural features of the 5′ UTR of HCV.

To test our hypothesis that miR-122 alters the architecture of the HCV IRES, as well as to better define the HCV targeting elements required by miR-122, we devised a new SHAPE-based method of interrogating RNA secondary structure at single nucleotide resolution. The goal of this new method was to make high-resolution RNA secondary structure mapping simple and fast.

The most advanced method for mapping RNA structure at single-nucleotide resolution is the SHAPE (Selective 2′-Hydroxyl Acylation analyzed by Primer Extension) method devised by Weeks and colleagues. This method has been well validated, the most striking example of which was the accurate prediction of the entire 16S ribosomal RNA secondary structures. SHAPE has recently been used to determine the architecture of the entire HIV genome. In this method, the RNA of interest is first interrogated by addition of N methylisatoic anhydride (NMIA), which preferentially acylates conformationally flexible nucleotides at the ribose 2′-OH position. During subsequent reverse transcription, these 2′-O-adducts result in pauses that can be detected by using fluorescent-labelled primers and separation by capillary electrophoresis. One of the largest bottlenecks in obtaining SHAPE data is the extensive data processing that is required, which currently may take an expert user up to 1-2 hours per data set. Visual inspection and manual data manipulation play a significant role in this data processing.

We have devised a series of strategies that significantly reduces the complexity and time requirements of SHAPE. First, the intricacies (and cost) introduced by using multiple fluorophores have been eliminated by using a single fluorophore-labelled primer and multiple capillaries, rather than using multiple fluorophores sets in each capillary. We have therefore named this technique, single fluorophore SHAPE (sf-SHAPE). sf-SHAPE is a fragment analysis method that uses an internal standard to allow data from one capillary to be directly compared to data from another, with high precision. Commercial software (Peak Scanner, ABI), as well as custom-designed software (CAFA)₁₁, which assign fragment lengths to peaks using an internal standard and integrates each peak area, are freely available.

We have designed a program entitled Fast Analysis of SHAPE Traces (FAST), which then takes the output from Peak Scanner and automatically (a) corrects for signal decay; (b) corrects for differences in signal strength among samples; and (c) assigns the determined SHAPE reactivity to the appropriate nucleotide, using a ddNTP sequencing ladder and the local southern method. This last step is non-trivial: although the use of an internal standard allows nucleic acid fragments to be assigned comparable lengths, these fragment lengths are not perfectly equivalent to true nucleotide (NT) position. FAST is able to generate SHAPE reactivity data from electrophoretic traces in as little as 5-10 minutes, as opposed to hours, with high experimental reproducibility (R₂=0.93).

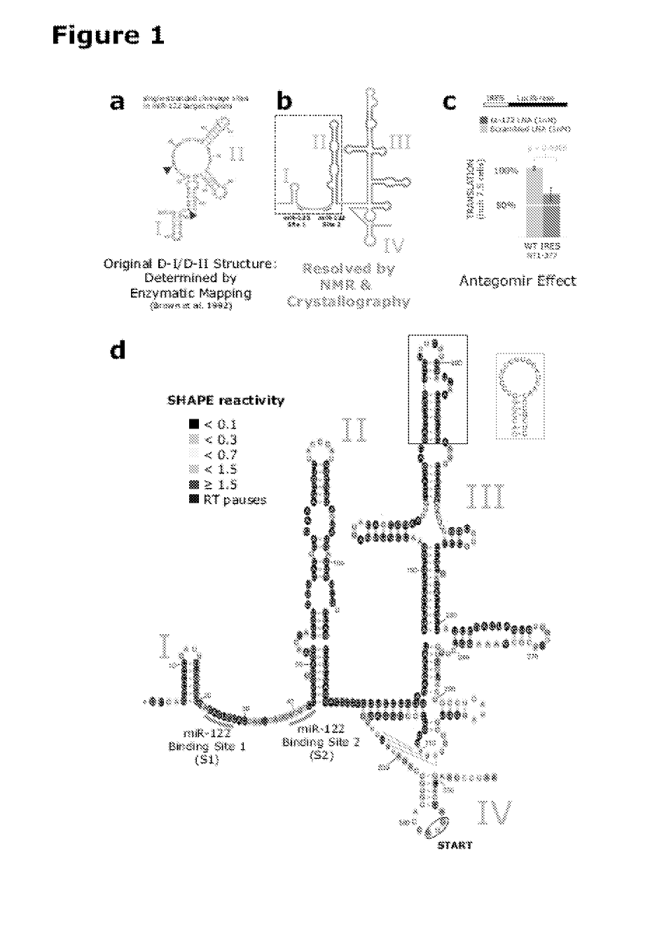

The HCV 5′ UTR is composed of four domains. The two miRNA target sites (51 and S2) occur in the region between domains I (D-I) and II (D-II). Domains II-IV define the HCV IRES. The most accurate data on the structure of the HCV IRES to date comes from NMR and crystallographic studies (FIG. 1b). Enzymatic mapping has resulted in notable inaccuracies, as evident in the original D-I/D-II structure (FIG. 1a). We began our experiments by confirming that the construct to be analysed, which consisted of the first 377 NTs of HCV, was sensitive to miR-122. This construct includes the entire 5′ UTR of HCV plus the first 36 NTs of the core gene. An in-frame firefly luciferase gene was then placed downstream, to assess translation activity (FIG. 1c). Co-transfection of this construct along with a locked nucleic acid (LNA) against miR-122 (antagomir) resulted in a ˜40% reduction in translation, compared to cotransfection with a control, scrambled sequence LNA. No differences in cell toxicity or transfection efficiency were observed. This degree of translation inhibition, due to depletion of endogenous miR-122, is similar to that previously observed and confirms that miR-122 can alter HCV translation. This data also confirms that domain VI (a part of the core gene not included in our construct) is not critical to miR-122 regulation.

Next, we used sf-SHAPE to determine the secondary structure of the 5′ UTR of HCV (NT1-377), which is illustrated in FIG. 1d. This model was created by using sf-SHAPE determined reactivities as nearest-neighbour free-energy change parameters in RNAstructure. SHAPE reactivities are normalized to a scale of 0 to ˜1.5, with reactivities above ˜0.3 generally being associated with nucleotides constrained by base pairing or other interactions.

Consistent with the high accuracy of SHAPE, we found that our secondary structure model matches the sub-domains of the HCV IRES resolved by NMR and crystallography studies (FIG. 1b). The one minor exception is NTs 227-229, which are found at the four way junction of D-III. These three nucleotides were found by sf-SHAPE to be flexible (yellow), whereas in the crystal structure they are double stranded. We suspect that this discrepancy is the result of the fact that the crystal structure model was solved using an HCV sequence with an artificially introduced GAAA hairpin near these nucleotides. As previously observed, SHAPE is also more accurate than enzymatic mapping (FIG. 1a.) This model also proposes a new structure for the apex of D-III. Finally, this model reveals that the second miR-122 binding site (S2) is single-stranded and accessible.

Target site accessibility has been shown to be critical for miRNA function. Therefore, this accessibility was expected, given the strong genetic evidence for interaction of miR-122 at this second sites. The SHAPE reactivity for the first miR-122 target site (51), however, suggests that it is composed of a mixture of double-stranded nucleotides (black) and nucleotides that cause the reverse transcriptase (RT) to pause and fall off, even from the unmodified, control RNA (blue). Since the identical target sequence at S2 does not cause the RT to pause, we speculated that it was not the sequence itself, but the local environment in which it appears that caused RT termination. This is consistent with the observation that the pause sites extend beyond the S1 target site. We therefore hypothesized the existence of a local, stable, highly structured RNA motif that prevents RT read-through. An alternative explanation would be that the pauses were caused by RNA degradation.

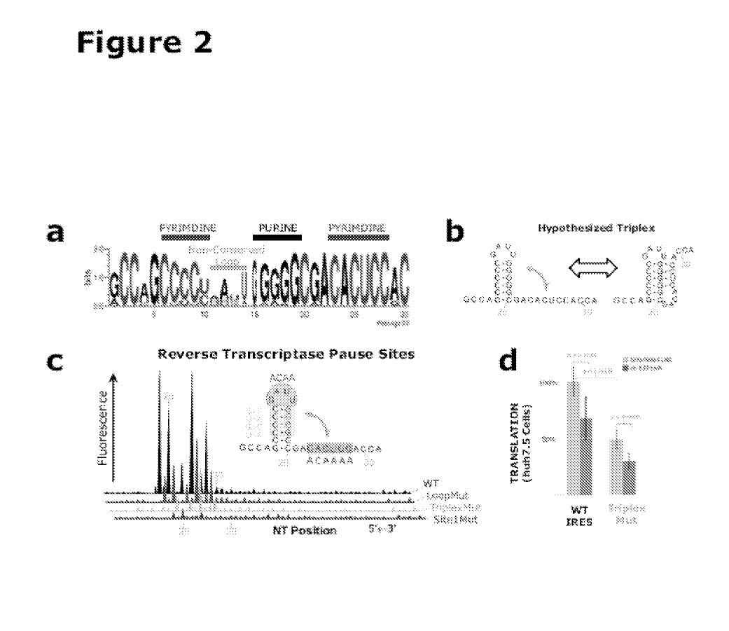

To explore what potential RNA motif could exist, we generated an alignment of >300 5′ UTR sequences from the European HCV database. This alignment revealed a pyrimidine-purine-pyrimidine (py-pur-py) signature that is highly conserved across all six HCV genotypes. The loop region of D-I was also found to be notably non-conserved (FIG. 2a), which argued against an H-type pseudoknot structure. We therefore hypothesized the existence of an RNA triplex (FIG. 2b), based on the fact that RNA triplexes are highly stable and are characterized by a py-pur-py signature. To test this hypothesis, we mutated the S1 target site (Site1Mut) and the stem sequence of D-I (TriplexMut), anticipating that both of these mutations should unfold the triplex and eliminate the pauses found at the S1 site. As a control, we also mutated the loop of D-I (LoopMut, FIG. 2c). In this experiment, the RT pauses and falls off only at regions of highly-stable RNA structure (equivalent to the control arm used in our structure analyses).

Mutation of the S1 target sequence (Site1Mut) completely eliminated RT pausing (red), consistent with the removal of a local RNA structure. Alteration of the D-I stem sequence (TriplexMut), which disrupts the py-pur-py motif, also eliminated nearly all the pause sites in the downstream S1 region (light blue). This suggests that the D-I stem interacts with the S1 region, since mutation is able to relieve the pausing that normally occurs there. Mutation of the loop of D-I did not abrogate pausing, although the extent of pausing at each site was diminished (pink). This suggests that the loop sequence is not critical to the formation of the putative triplex. Thus, genetic, structural, and mutagenic studies support the existence of a highly-structured RNA motif at the 5′ terminus of HCV that has the hallmarks of an RNA triplex.

Next, we explored the functional importance of this structure with respect to HCV translation. Prior groups have observed that mutation of the D-I stem, but not the D-I loop, reduced translation₂₂. We introduced the above triplex mutation (TriplexMut) into our IRES-luciferase construct to test the effect of maintaining the D-I duplex structure, but disrupting the potential triplex structure. We found that, consistent with prior observations, disruption of the triplex motif resulted in a ˜50% decrease in translation (FIG. 2d). No differences in cell toxicity or transfection efficiency were noted. By way of comparison, Puglisi and colleagues found a ˜80% decrease in translation when all of domain II was removed. Thus, although D-I is not part of the classically-defined HCV IRES, disruption of the putative triplex in D-I disrupts translation to a degree roughly similar to removal of D-II.

We also observed that the TriplexMut construct remained sensitive to miR-122 knockdown by an antagomir. This suggests that, miR-122 does not necessarily enhance translation by disrupting the triplex structure. Conversely, it also suggests that the triplex, or arguably the D-I sequence itself, play a role in translation independent of containing the S1 target site. Since duplexes (stems) are typically thought of as acting as inert scaffolds that position loop regions, our proposal that this stem is actually part of a triplex would explain why HCV translation would be sensitive to stem sequence. Finally, a review of the 5′ termini of other members of the flaviviridae family failed to demonstrate a py-pur-py signature in any other viral member, including its closest relative, GBV-B. This complex RNA motif therefore appears to have evolved specifically in HCV.

To our knowledge, triplexes have not previously been described in the non-coding region of viruses, although more typical pseudoknots found at the 5′ terminus of viruses have been found to be important for replication. It is noteworthy that D-I is known to be critical for HCV replication₂₂. Thus, it is possible that this putative triplex may also play a direct role in replication. Intriguingly, a recent report found the S1 site, which is part of the triplex, to be more critical for high viral titers than the S2 site.

We next explored the effect of miR-122 on the HCV IRES structure. In this experiment, the HCV IRES structure was determined in the presence of miR-122 as well as a control miRNA, miR-124. The IRES structure was also determined in the presence of a mutant miR-122, in which the seed region of miR-122 was retained (NT 1-9), while the remainder of the miRNA was replaced by the end of miR-124 [miR-122(seed) with miR-124(tail)].

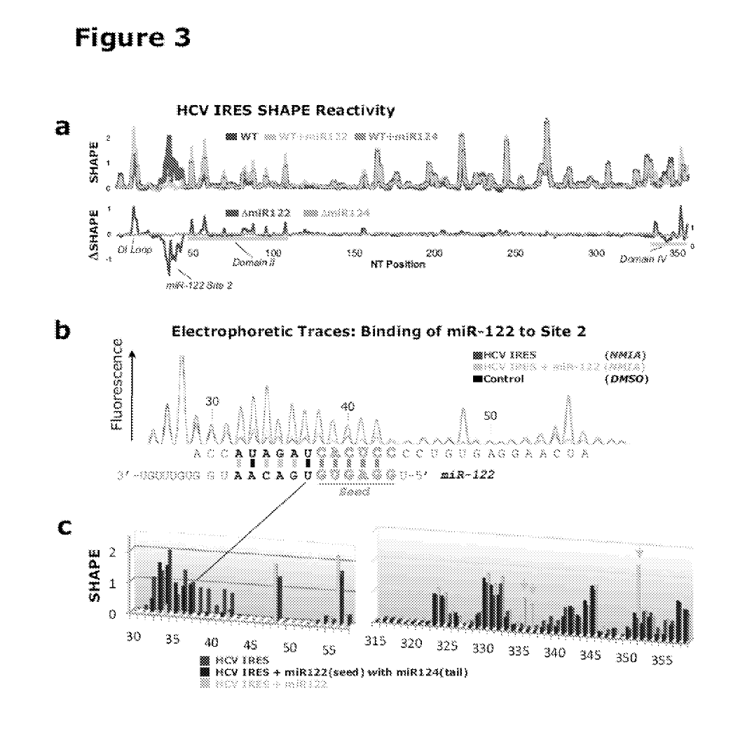

The SHAPE reactivity values for the HCV IRES (WT, red), in the presence of miR-122 (green) versus miR-124 (grey) are shown in FIG. 3a. The addition of control miR-124 had no effect on IRES structure; this is most clearly illustrated in the bottom panel of FIG. 3a (orange), in which the SHAPE values, with and without miR-124, were subtracted from one another. Conversely, miR-122 induced a number of changes in the HCV IRES structure (subtraction trace, purple). While the entire D-III region is unchanged, the flexible D-I loop and other flexible nucleotides in D-II have enhanced SHAPE reactivity. As D-II is bound by the 40S ribosomal subunit, it is possible that a less compact, more flexible D-II structure is advantageous to this interaction; however, the significance of increasing already high SHAPE reactivity values has not yet been well-delineated.

Of greater immediate interest is that upon addition of miR-122, (a) the S2 target site experiences a significant reduction in flexibility, consistent with duplex formation at this site, and (b) the Domain IV stem becomes more flexible, suggestive of a change in conformation from double-stranded to single-stranded. FIG. 3b is a close-up of the electrophoretic trace of the S2 target site. Even from this raw data, it is clear that miR-122 reduces the flexibility of HCV nucleotides 32-42. While seed base-pairing was expected to be observed (NT 38-42, grey), the decrease in the signal of HCV NTs 32-37 is noteworthy, since these nucleotides fall outside the complementary seed region. A number of possible explanations for changes in reactivity outside the complementary seed target can be postulated. First, it is possible that miR-122 directly interacts with these nucleotides, forming a series of canonical (black) and non-canonical (orange) interactions with the HCV IRES, in addition to seed base-pairing (FIG. 3b). Second, it is possible that these interactions are non-specific and simply the result of the tail of miR-122 indiscriminately overlapping this region. Third, it is possible that binding at the seed site indirectly induces a local conformational change that reduces the reactivity of NTs 32-37, but that direct interaction between the tail of miR-122 and these nucleotides does not occur.

The structure of the HCV IRES determined in the presence of the mutant miR-122, which has the seed sequence of miR-122 but the tail of miR-124, allowed us to distinguish among these possibilities. The calculated SHAPE reactivities for this region of HCV are shown in FIG. 3c, left. While both the WT miR-122 (green) and the miR₁₀ 122 tail mutant (blue) bound to the S2 target of the IRES (NT 38-42), only the WT miR-122 results in binding at NTs 32-37. From this we can conclude that binding at the seed sequence is not sufficient to result in a change in reactivity for NTs 32-37; nor is it the case that the mere presence of nucleotides downstream of the seed sequence is able to alter the conformational state of these nucleotides. Thus, it appears that miR-122 makes a number of non-canonical interactions (NCIs) with its target.

A 6-NT seed site interaction with its target may be insufficient for a stable, specific interaction. For this reason, it has been hypothesized that miRNAs are presented to their targets in a pre-formed semi-helical state by the Ago protein, which enhances targeting₁. Our data suggests the specificity and stability of miR-122 targeting to HCV is enhanced by a series of non-canonical interactions outside the seed site, which occur in the absence of protein factors. To our knowledge, this is the first demonstration of non-canonical interactions being described as part of miRNA targeting, and reveals the power of SHAPE to define the extent of direct binding by a miRNA to its target site.

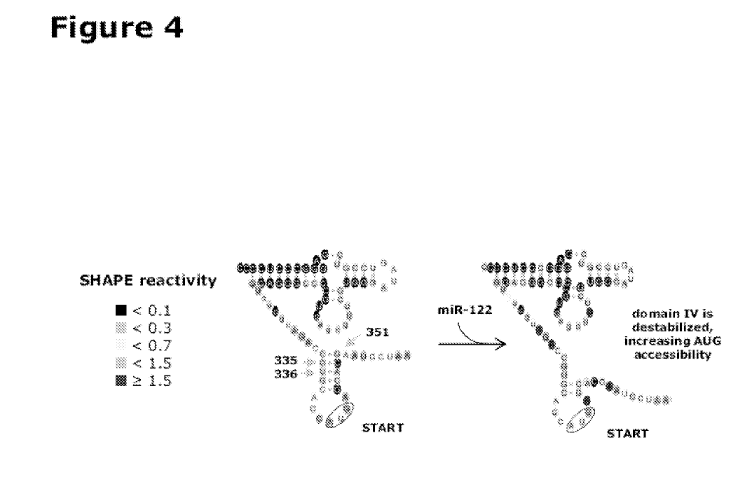

We next explored the effect of miR-122 binding on domain IV structure, the region of the IRES that contains the AUG start codon (FIG. 3c, right). It has previously been observed that mutations that decreased the stability of the D-IV stem enhanced IRES translation₂₇. Such destabilization has been proposed to allow for proper positioning of the AUG codon in the 40S ribosomal subunit. These observations raise the question of how such destabilization can, in practice, be achieved. In our HCV IRES structure, D-IV forms a single stem-loop structure (FIG. 1d). Addition of miR-122 resulted in significantly increased flexibility in this region, most notably for nucleotides 335, 336, and 351 (FIG. 3c, right). Nucleotides 335 and 336 compose part of the stem of D-IV. Their increased flexibility suggests stem destabilization by miR-122 (FIG. 4). As noted previously, stem destabilization is known to enhance IRES translation.

We also note that although the miR-122 mutant with the tail of miR-124 is able to bind to its target site, (FIG. 3c, left), it does not induce changes in the reactivity of D-IV (FIG. 3c, right). This suggests that the destabilization that occurs in domain IV requires the tail of miR-122, and argues against the possibility that binding of miRNA seed nucleotides is sufficient to cause this architectural rearrangement. Since our data suggests that the S1 site is likely occupied by interactions with the D-I stem, it followed that miR-122-induced changes in D-IV were likely caused entirely by binding of miR-122 to S2. We confirmed this by performing structural studies on a 5′ UTR construct whose S1 was mutated to prevent binding (Site1 Mut). As expected, despite the inability of miR-122 to bind at the S1 site, binding at the S2 site was sufficient to cause the same rearrangement of nucleotides in D-IV that were observed in the WT construct. Thus, the conformational changes observed at the AUG start site are mediated by the tail of the miR-122 that binds to S2.

What, then, are the structural effects of miR-122 binding to S1? Strong genetic evidence exists demonstrating miR-122 binds to S1 within cells_{3,5}. The presence of a triplex suggests the existence of a mechanism by which this structure is unwound, allowing for miR-122 binding. One can speculate on the possibility that unwinding is accomplished by a component of RISC. To confirm that these structural insights into miRNA-mediated HCV translation were neither an artifact of the construct used nor specific to HCV genotype 1, we performed these same structural analyses on a genotype 2 RNA construct that consisted of the first 990 NTs of HCV. This construct included the HCV IRES and the entire core gene. We observed the same findings (data not shown). We therefore conclude that the structural observations we have made are valid for both genotypes 1 and 2. As these genotypes span the evolutionary spectrum of HCV, this suggests that our results are generalizable to all HCV genotypes.

In summary, we propose a model in which miR-122 enhances viral translation, in part, by using its tail to destabilize D-IV (FIG. 4). This destabilization allows for improved positioning of the AUG start site in the 40S entry channel upon recruitment of the ternary complexes, consistent with the observation that miR-122 accelerates 48S complex formations. In this model, the first miR-122 binding site is part of a putative RNA triplex, which likely requires unwinding prior to binding, while the second target site is always accessible and binds to miR-122 via a number of noncanonical interactions.

### Methods

SHAPE performed as previously described by Mortimer & Weeks (2009) Nature protocols 4, 1413-1421; and Wilkinson et al. (2006) Nature protocols 1, 1610-1616 except: RNA purification after acylation and removal of miRNA before RT by RNA C&C (Zymoresearch); DNA purification by size exclusion (Sephadex G-50 resin); 1 pmol RNA used in sequences reactions; fragment analysis protocol (GeneScan/ROX 500 standard/50 cm capillary/POP6/voltage 15 kV/T=60° C./injection time=15 s (3100 ABI); 6-FAM labelled primers), and PeakScanner/FAST analysis. IRES folding29, (100 mM NaCl; 2.5 mM MgCl), alamar blue assay and qRT-PCR were performed as previously described₃₀. Luciferase assay performed as described₃₀, except co-transfection (HCV/LNA) was by lipofectamine 2000 (3.5×10⁵ huh 7.5 cells/well), with translation measured at 4 hours. LNAs purchased from Exiqon (Scramble-miR/hsa-miR-122 kd2). Sequence logo: RNA graphics were drawn using RNA Viz. P-values (two-tailed unpaired t-test) calculated by InStat 3 (Graphpad).

PeakScanner Processing. PeakScanner 1.0 Parameters: smoothing=none; window size=25; size calling=local southern; baseline window=51; peak threshold=15. Fragments 250 and 340 were excluded from the ROX500 standard.

FAST Algorithms. Automated Scaling: FAST automatically determines the scaling factor (k) that corrects for gain differences between different experimental arms and the control data set by empirically testing values of k, to determine the value of k such that M (Eq. 1) approaches zero.

M=Median(kEⱼ−Cⱼ) (1)

In this equation, E is the experimental dataset of peak areas and their positions; C is the control (DMSO) dataset of peak areas and their positions; j is each individual peak position. The median operator is notably insensitive to how data is distributed. Thus, as the median of the difference between experimental and control data sets approaches zero, the result in nearly perfect overlap among the smaller, noise peaks in both data sets. This algorithm is quite robust: nearly identical SHAPE profiles were obtained even when the ratio of experimental and control samples was deliberately and significantly varied. Cech and colleagues also developed a computerized normalization algorithm; but in this algorithm an a priori cut-off of the 10% of most unreactive peaks is used to normalize different datasets.

One minor modification of Eq. 1 is to redefine J as the subset of peak positions that satisfy Eq. 2. In this case, X is then the set of all peak positions.

J={jεX∥E(j)−C(j)|<A} (2)

This redefinition has the practical effect of limiting the values included in the median calculation to those in which the difference between the experimental peak area and control peak area is “small”, as defined by A. If A is set to a large number, then all peak positions are included, resulting in the original calculation. The benefit of Eq. 2 is that for excellent quality samples in which the experimental and control data sets are quite alike, Eq. 2 limits potential over-fitting. This reduces the likelihood that a nucleotide is not assigned a SHAPE value, because it has a significantly negative value. Thus, as A is made smaller and smaller, the number of unassigned nucleotides, if existent, can be potentially reduced in number. However, there is a lower limit for A below which the number of peak positions included in set J becomes too small to result in a meaningful fit.

Automated Signal Decay Correction:

Signal decay results from multiple hits by the acylation chemical or salt/primer concentration differences that affect uptake by the electrokinetic injector. The equation used to correct for this decay was described by Dr. Weeks as:

D=Ap^{elution time}+C (3)

In this equation, D is the correction factor by which each peak area is divided; A and C are scaling factors for the initial and final trace intensities, respectively; and p is the probability of extension. Since our fragment analysis technique assigns a fragment length to each peak, “elution time” is replaced by fragment length.

The goal of this equation is to have the resultant peaks display no bias in their intensities (e.g. peaks at the start of the trace are not higher than those at the end of the trace). This is visually achieved by applying D to the data until the peak heights are generally uniform across the trace (i.e. high peak at the start of the trace are equal in height to high peaks at the end of the trace, etc, etc).

In FAST, this visual inspection is replaced by empirically determining the value of p (Eq. 3) such that m (Eq. 4) approaches zero, when all the peak areas in a given data set are divided by D.

(Peak Area)ⱼ/D=m(Fragment Length)+b. (4)

Eq. 4 describes a simple line, where m is the slope, b is the intercept, and J is the set of all peaks in the data set. As m approaches zero, the line becomes flat, resulting in the removal of signal decay from the electrophoretic trace. In our experience, we have found signal decay to be highly sensitive to the cleanliness of the sample, and have found desalting/primer removal by centrifugation though size exclusion beads to be highly advantageous.

Automated Assignment of Peaks to Nucleotide Positions:

The use of an internal standard allows nucleic acid fragments to be assigned comparable lengths according to a common scale, and thus allows for comparisons among different capillaries and different experimental arms. Such assigned fragment lengths, however, are only closely related to, but well-know not to be perfectly equivalent to, true NT position. This is illustrated in Supplemental FIG. 4a, in which fragment length and nucleotide position are plotted against one another. Macroscopically they appear identical (light grey). Upon close inspection, differences of up to nearly 4 NTs between the assigned fragment length and the actual NT position exist (black line, open circles). For this reason, the fragment analysis method alone is insufficient to accurately assign SHAPE reactivities to specific nucleotides. Such assignments require an external standard. For sf-SHAPE, like standard SHAPE, a sequencing ladder generated by performing the reverse transcriptase reaction in the presence of a ddNTP is used as the external standard.

This ladder can then be assigned true nucleotide positions, through an iterative decision algorithm. For example, if ddGTP is used to generate the sequencing ladder, the algorithm takes advantage of the fact that the sequence of the RNA is known and that each successive peak in the ladder must correspond to the next G in the sequence. Missing peaks or aberrant peaks can be corrected for by assuming that the offset between fragment length and true nucleotide position, while changing, changes in a series of incremental steps (Supplemental FIG. 4a). Thus, if the delta-delta between successive offsets is large, the program recalculates nearby assignments until such delta-deltas are all small.

Once the peaks in the ladder have been assigned true nucleotide positions, these assignments can then be used to assign true nucleotide positions to all peaks in the experimental or control arms, using the local southern method⁴. Briefly, a first curve is generated by using two ladder peaks below and one ladder peak above each peak of interest. The peak of interest is then assigned a position based on its fit to this first curve. A second curve is generated using two ladder peaks above and one ladder peak below the same peak of interest. The peak of interest is then assigned another position based on this second curve. The two determined positions are then averaged to arrive at the final assigned position for the peak. The resultant average in nearly all cases can simply be rounded to result in the integer value for the true nucleotide position. The FAST program institutes multiple checks to ensure and rectify situations in which peaks have been assigned to the same nucleotide, and that every peak has been assigned to a nucleotide position. It has not been found necessary to generate more than one sequencing ladder to make these assignments.

### Materials

Unless otherwise indicated, published materials used for SHAPE³. IRES constructs: GN1b, PCR product from Bart79I, originally from Charles Rice; GN2 PCR product from isolate J6. RNA: generated by in-vitro transcription, using T7 MEGAscript; RNA for SHAPE purified by MEGAclear, with purity and length verified by capillary electrophoresis; RNA for transfection purified by LiCl precipitation, with purity and length verified by agarose gel.

Primers. miRNA: miR-124: UAAGGCACGC GGUGAAUGCC; miR-122-RNA: UGGAGUGUGA CAAUGGUGUUUG; mir122with124tail: UGGAGUGUGC GGUGAAUGCC; SHAPE primers: 6FAM-CCAGGCATTG AGCGGGTTGA TCCAAG; 6FAM-CCTGCGTGCA ATCCATCTTG; Mutation primers: TriplexMut: GGAAGGAAGG AAGAGATAAT ACGACTCACT ATAGCCAGCT GTGGATTCAC AGCGACACTC CACCATAGAT CACTCCCCTGTG; LoopMut: GGAAGGAAGG AAGAGATAAT ACGACTCACT ATAGCCAGCC CCCACAAGGG GGCGACACTC CACCATAGAT CACTCCCCTGTG; Site1 Mut: GGAAGGAAGG AAGAGATAAT ACGACTCACT ATAGCCAGCC CCCGATTGGG GGCGAACAAA AACCATAGAT CACTCCCCTG TGAGGAACTAC; PCR Template primers: (GN1) GTCTGACGCTC AGTGGAACGA AAACTCACG; CCTGCGTGCA ATCCATCTTG TTCAATCATG; (GN2) CATTCAGGCT GCGCAACTGTTG; GACAGCATTA CCTGGCAGCTCC.

### Example 2

### High-Throughput, High Resolution RNA Secondary Structure Determination

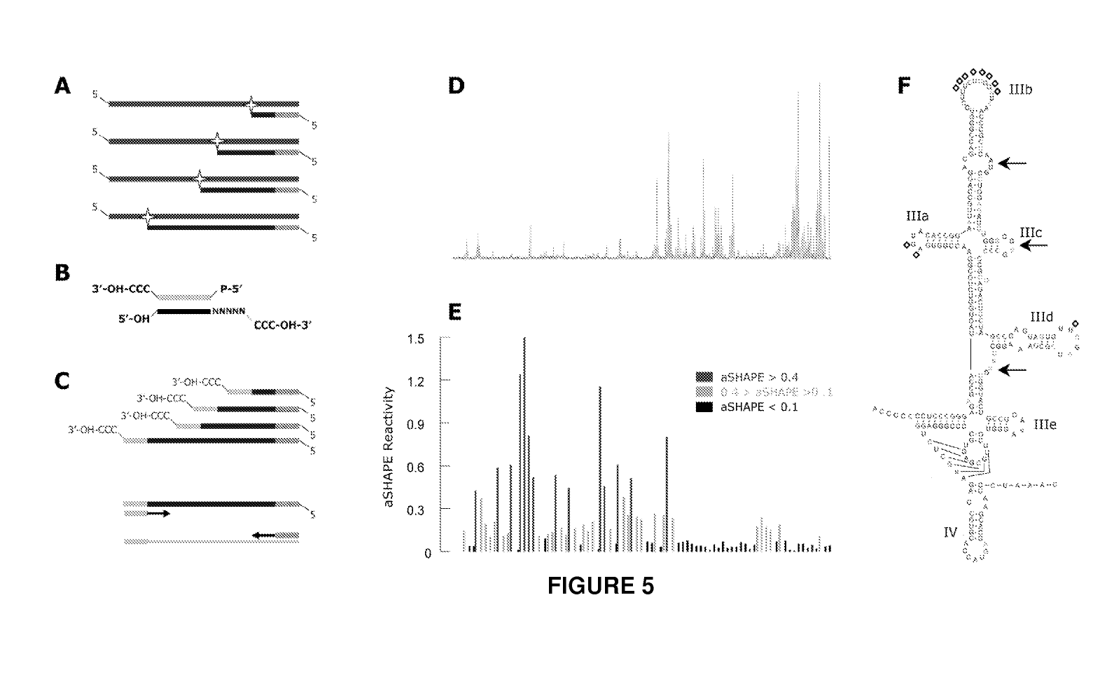

As shown in FIG. 5, target RNA is used in its naturally folded state, and flexible 2′ hydroxyl groups are acylated under single hit conditions. The result is a population of RNAs, each of which has been acylated at a single position (A), where the target RNA (red) is schematized to be chemically modified (star) at nucleotides with local flexibility, under single hit conditions. During reverse transcription (RT), the transcriptase falls off the RNA at each acylation site, resulting in a pool of DNA molecules (blue) whose lengths correspond to sites of RNA flexibility. An adaptor (B) is used to add a 3′ primer site (orange) to the end of reverse transcribed DNA (C). This adaptor has a poly-N overhang that acts as a bridge and allows the adaptor to bind to any 3′ DNA termini. Ligation is then carried out using T4 DNA ligase. To eliminate unwanted ligation products, only one 5′ phosphate site exists on the adaptor and all 3′OH sites in the adaptor have been replaced by three-carbon moieties that prevent ligation or extension. After ligation, every reverse transcribed DNA molecule contains the same known sequences at the two ends. This allows for PCR amplification of this pool of DNA fragments using a fluorophore-labeled oligonucleotide (green) complimentary to the adaptor and the original RT primer (light blue) (C, lower panel). The resultant PCR fragments are then separated using capillary electrophoresis (D) and the signal is then processed (E). Higher reactivity corresponds to increased nucleotide flexibility, typically associated with non-based paired nucleotides. Positions of decreased reactivity are also informative and denote structural rigidity. These values can be used as pseudo-free energy constraints in the RNAstructure folding algorithm. The resultant predicted RNA secondary structure is shown in (F).

### Example 3

### Determination of the In Vivo RNA Secondary Structure of the HCV Genome and its Role in Viral Replication and Virion Production

As a first step in identifying novel RNA secondary structures within the HCV genome critical to the virus, the in vivo RNA secondary structure of the HCV genome is determined.

aSHAPE and FAST are used to determine the secondary structure of the HCV genome as it exists in cells. Evolutionarily conserved structures are identified by covariation and synonymous/non-synonymous sequence alignment analysis. Mutational analysis will allow characterization of the role of genomic RNA structure in viral replication and virion formation. It is determined if a correlation exists between the location of RNA structures in the genome and the location of the inter-domain loops of viral proteins. Such a correlation, in conjunction with the footprinting of ribosomal pauses, would support the hypothesis that RNA structure promotes proper protein folding by causing the ribosome to pause after each domain of a protein is translated. Thermodynamic signatures suggests that RNA structure is encoded throughout the HCV genome, in contrast to that of the HIV genome. Additionally, knowledge of the entire secondary structure of the HCV genome allows this structural information to be incorporated into HCV evolutionary models. Such evolutionary information will help inform the rate at which drug resistance is likely to develop against RNA-targeted therapeutics. Finally, examination of the relative prevalence of UU and UA dinucleotides in single-stranded versus duplex regions will allow for a preliminary assessment of the role of RNA structure in HCV evasion from RNase L, a host antiviral factor that only cleaves single stranded UU and UA dinucleotides.

Some regions of the HCV genome may exist in multiple structural states, which may result in ambiguous RNA secondary structure models for these regions. Determining the HCV genomic RNA secondary structure in different biological contexts (from virions, in vitro, and in vivo) will resolve such ambiguities and provide insight whether such multiple structural states are due to the cellular environment or are an intrinsic property of the RNA. Ultracentrifugation may be used to isolate genomic RNA from the cytoplasmic membranous webs upon which replication occurs. Finally, HCV genomic RNA also serves as mRNA. Thus, mapping in vivo structure in the presence and absence of ribosomal inhibitors (as used in ribosomal footprinting), will be informative and may reduce any ribosome-induced RNA structural heterogeneity.

### Example 4

### Disruption of Newly Identified RNA Secondary Structure Elements by Synonymous Mutations, In Order to Test the Role of Individual RNA Secondary Structure Elements in HCV Replication and Virion Production

NA secondary structures are disrupted to test their role in HCV replication and virion formation. WT and HCV mutant viruses are generated, transcribed, and transfected into huh7.5 cells as previously described. qRT-PCR is used to determine cellular and virion copy number. Limiting dilution assays are used to determine the 50% tissue culture infectious dose (TCID₅₀) for each mutant.

Many of the RNA secondary structures are expected to be critical to either HCV replication and/or virion formation. RNA structures found to be unimportant to the virus in cell culture may nevertheless be relevant to HCV pathogenesis in humans, which may be tested with small animal models of HCV.

To ensure that RNA structure disruption does not affect encoded proteins, only synonymous changes will be made. One alternative approach is to select the subset of the most complex RNA structures (as denoted by multi-way junctions) for mutagenic study. A second alternative is to use megaprimer PCR mutagenesis, which is rapid and easy to scale up. The limited fidelity of thermostable polymerases, however, may accidentally introduce unwanted mutations, making this alternative more comprehensive, but which will also result in a higher false-positive rate.

### Example 5

### Identification of Novel, Small Molecules Active Against HCV by Screening Compound Libraries for Small Molecules that Alter HCV RNA Structure

The LoPac (Sigma/1280 compounds) and ICCB (Biomol3/480 compounds) validation libraries contain a diverse collection of known bioactives. Compounds in these libraries are screened for HCV RNA binding activity as measured by sf-SHAPE.

Compounds that alter HCV RNA structure are identified and utilized as lead compounds for generation of libraries of novel chemical derivatives. As a positive control the small molecule designate “13,” which is known to bind the HCV IRES and thereby inhibit HCV replication is screened. Cross-screening is performed against non-HCV RNA structures (EMCV IRES; delta virus ribozyme) to ensure the specificity of any candidate compounds.

PubChem is used to identify derivatives of lead compounds that possess structure similarity. A library of such compounds is assembled (from commercial suppliers and with the aid of synthetic medicinal chemists), and their HCV RNA binding affinity tested using sf-SHAPE. In this manner, Structure-Activity Relationships (SAR) for each of the lead compounds is determined. Cross-screening for specificity will again be performed.

The expected result is a set of compounds that bind to a given target HCV RNA structure with nanomolar dissociation constants (K_{d}), but which do not bind or have high dissociation constants for the non-HCV RNA structures tested.

Candidate small molecule derivatives that have high HCV RNA binding affinity are screened for their ability to inhibit HCV replication and virion formation in cell culture. Wild-type HCV genomic RNA capable of replication and virion production is transfected into huh7.5 cells. Transfected cells are treated with each compound of interest (at a range of dilutions) for 72 hours (in which the media is replaced daily with media containing fresh drug solutions). qRT-PCR will then be used to assess virion production and viral replication, and calculate half-maximal inhibitor concentrations (IC50). AlamarBlue (Invitrogen), a colorimetric indicator of cell toxicity, is used to determine the half maximum cytotoxic concentration (CC₅₀).

These studies are used to identify compounds that inhibit HCV replication and virion production and have therapeutic potential. Preferably, such compounds will have high potency (low IC₅₀ values) and low toxicity (high CC₅₀ values). Compounds that inhibit viral replication will result in low viral copy numbers in both cells and media; compounds that inhibit only virion production will result in lower viral copy numbers in the media only.

Drugs that target different stages of the HCV life cycle may exhibit synergism. Thus, even if an individual compound exhibits limited anti-HCV activity, a combination of inhibitors that target both viral replication and virion formation may be found that result in high potency. Animal studies may be used to further evaluate toxicity and efficacy.

1. A method for the determination of higher structure in a polynucleotide of interest that is at least partially single-stranded, the method comprising:
contacting said polynucleotide of interest with a modifying agent that alters the polynucleotide at sites having a defined secondary structure, wherein the modification is such that it causes a halt in a polymerization reaction when polynucleotide is used as a template for polymerization to generate a set of modified polynucleotides;
polymerizing a second polynucleotide that is complementary to said modified polynucleotide, wherein polymerization is truncated at a modified nucleotide, to generate a set of polymerization products that vary in length;
ligating said second polynucleotides to an adapter, wherein said adapter comprises a sequence for priming amplification;
amplifying a polynucleotide with a primer set complementary to said adapter and to a second, defined position, wherein at least one primer of said set comprises a detectable label to generate a set of amplification products that vary in length;
determining the size and/or sequence of said set of amplification products, wherein each site of truncation corresponds to a site having a defined higher structure; and
correlating said sites of higher structure to provide a structure analysis of said polynucleotide of interest.

2. The method of claim 1, wherein the polynucleotide of interest is an RNA.

3. The method of claim 1, wherein said RNA is an mRNA.

4. The method of claim 1, wherein said RNA is a viral genome or fragment thereof.

5. The method of claim 1 wherein said polynucleotide is a DNA that is at least partially single stranded.

6. The method of claim 5, wherein said DNA is a viral genome.

7. The method of claim 1, wherein said modifying agent is a chemical agent.

8. The method of claim 7, wherein said agent is selected from N-methylisatoic anhydride (NMIA), 1-methyl-7-nitroisatoic anhydride (1M7), Benzoyl CN, dimethylsulfoxide (DMSO), diethylpyrocarbonate (DEPC), Pb²⁺, and Fe²⁺.

9. The method of claim 1 wherein said modifying agent in an enzyme.

10. The method of claim 9, wherein said enzyme is selected from RNAse T1 andra venom V1 nuclease.

11. The method of claim 2, wherein said polymerase is reverse transcriptase.

12. The method of claim 1 wherein said amplification is performed by PCR.

13. The method of claim 1, wherein the size of said set of amplification products is determined by capillary electrophoresis.

14. The method of claim 1, wherein said set of amplification products are sequenced.

15. The method of claim 1, wherein said polynucleotide of interest is isolated.

16. The method of claim 1, wherein 1, wherein said polynucleotide of interest is present in a complex population.

17. The method of claim 16, wherein said polynucleotide of interest is present in an intact cell.

18. The method of claim 1, wherein said polynucleotide of interest is associated with at least one macromolecule.

19. The method of claim 18, wherein said macromolecule is a protein.

20. The method of claim 19, wherein said polynucleotide of interest is present in a virion.

21. The method of claim 1, wherein the higher structure is secondary structure.

22. The method of claim 1, wherein the higher structure is tertiary structure.

23. The method according to claim 1, further comprising contacting said polynucleotide of interest with a candidate agent; and comparing the analysis of secondary structure in the absence and presence of said agent.

24. The method of claim 23, further comprising determining whether an agent modifies the structure of said polynucleotide.

25. The method of claim 24, further comprising determining the effect of a library of candidate agents.

26. The method of claim 25, further comprising testing said agent for activity in altering a function of said polynucleotide of interest.

27. The method according to claim 1, further comprising mutagenizing said polynucleotide of interest; and comparing the analysis of secondary structure in the absence and presence of said mutagenesis.

28. The method of claim 27, further comprising determining whether a mutation modifies the structure of said polynucleotide.

29. The method of claim 28, further comprising determining the effect of a library of mutations.

30. The method of claim 25, further comprising testing said mutation for activity in altering a function of said polynucleotide of interest.

31. The method of claim 1, wherein said step of correlating said sites of higher structure to provide a structure analysis of said polynucleotide of interest comprises:
correcting for (a) fragments not due to modification and (b) signal decay as a function of fragment length.

32. The method of claim 31, wherein signal decay is corrected for by dividing the signal of each fragment length by a correction factor (X), that is the result of exponential function such as: X=A(p)^{length}+B (eq 1).

33. The method of claim 32, wherein iterative inverse least-squares fit is used find a p value, which, when input into equation 1, results in slope m˜0.

34. The method of claim 32 wherein the data is normalized by Minimization of the Median.


SEQUENCE LISTING (18 sequences)

SEQ ID NO: 1 - RNA, 20 nt, Artificial Sequence
  Synthetic Primer
uaaggcacgcggugaaugcc

SEQ ID NO: 2 - RNA, 22 nt, Artificial Sequence
  Synthetic Primer
uggagugugacaaugguguuug

SEQ ID NO: 3 - RNA, 20 nt, Artificial Sequence
  Synthetic Primer
uggagugugcggugaaugcc

SEQ ID NO: 4 - DNA, 26 bp, Artificial Sequence
  Synthetic Primer
ccaggcattgagcgggttgatccaag

SEQ ID NO: 5 - DNA, 20 bp, Artificial Sequence
  Synthetic Primer
cctgcgtgcaatccatcttg

SEQ ID NO: 6 - DNA, 82 bp, Artificial Sequence
  Synthetic Primer
ggaaggaaggaagagataatacgactcactatagccagctgtggattcacagcgacactccaccatagatcactcccctgtg

SEQ ID NO: 7 - DNA, 82 bp, Artificial Sequence
  Synthetic Primer
ggaaggaaggaagagataatacgactcactatagccagcccccacaagggggcgacactccaccatagatcactcccctgtg

SEQ ID NO: 8 - DNA, 91 bp, Artificial Sequence
  Synthetic Primer
ggaaggaaggaagagataatacgactcactatagccagcccccgattgggggcgaacaaaaaccatagatcactcccctgtgaggaactac

SEQ ID NO: 9 - DNA, 30 bp, Artificial Sequence
  Synthetic Primer
gtctgacgctcagtggaacgaaaactcacg

SEQ ID NO: 10 - DNA, 30 bp, Artificial Sequence
  Synthetic Primer
cctgcgtgcaatccatcttgttcaatcatg

SEQ ID NO: 11 - DNA, 22 bp, Artificial Sequence
  Synthetic Primer
cattcaggctgcgcaactgttg

SEQ ID NO: 12 - DNA, 22 bp, Artificial Sequence
  Synthetic Primer
gacagcattacctggcagctcc

SEQ ID NO: 13 - RNA, 354 nt, Artificial Sequence
  Synthetic Primer
gccagcccccgauugggggccagacucgccauagaucacuccgcuggaggaacuacugucuucacgcagaaagcgucuagccauggcguuaguaugagugucgugcagccuccaggacccccccucccgggagagccaugaguggucugcggaaccggugaguacaccggaauugccaggacgaccggguccuuucuuggaucacaacccgcucaaugccuggagauuugggccugcccccgcgagacugcuagccgagugugguugggucgcgaaggccuguacugcccugauagggugcuugcgagugccccggagguccguagaccgugcaccaugagcaugaauaccuaa

SEQ ID NO: 14 - RNA, 30 nt, Artificial Sequence
  Synthetic Primer
gccagccccuaauugggggcgacacuccac

SEQ ID NO: 15 - RNA, 32 nt, Artificial Sequence
  Synthetic Primer
gccagcccccgauugggggcgacacuccagga

SEQ ID NO: 16 - RNA, 31 nt, Artificial Sequence
  Synthetic Primer
gccagcccccgauucgggcgacacuccacca

SEQ ID NO: 17 - RNA, 54 nt, Artificial Sequence
  Synthetic Primer
accauagaucacuccccugugaggaacuauguuugugguaacagugugagaggu

SEQ ID NO: 18 - RNA, 82 nt, Artificial Sequence
  Synthetic Primer
ccccugccggagcugccugauauggugcuugcaggugccccgggaggucucguagaccgugcaccaugagcuggaauccuaa